(19) United States
(12) Patent Application Publication
(10) Pub. No.: US 2002/0016539 A1
(43) Pub. Date: Feb. 7, 2002

(54) Method and apparatus for measuring and classifying optically observable changes in skin and mucous membrane

(76) Inventors: Bernd Michaelis, Biederitz (DE); Peter Albrecht, Magdeburg (DE)

Correspondence Address: Law Offices of Karl Hormann, 86 Sparks Street, Cambridge, MA, 02138-2216, US

(21) Appl. No.: 09/849,386

(22) Filed: May 3, 2001

(30) Foreign Application Priority Data
May 3, 2000 (DE) 100 21 431.2-35

(51) Int. Cl. A61B 5/05

(52) U.S. Cl. 600/407; 600/408; 600/476

(57) ABSTRACT

A method and an apparatus for measuring and classifying skin anomalies by taking at least one digital image of the anomaly and a digital color reference image of healthy skin in the vicinity of the anomaly, computing surface measurement data including three-dimensional coordinates and color values scaled to the reference image of the anomaly and classifying the anomaly in predetermined categories on the basis of the surface measurement data.

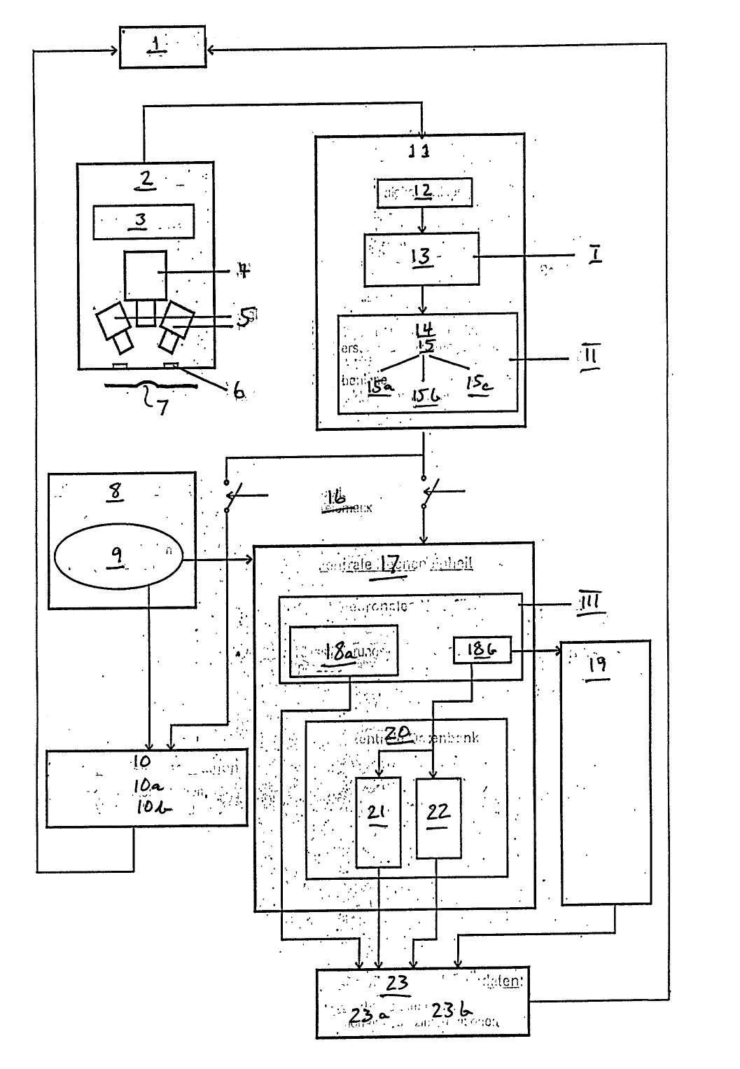

### BACKGROUND OF THE INVENTION

1. Field of the Invention

The invention, in general, relates to a method and to an apparatus for measuring and classifying, in predetermined categories, optically observable changes in the skin and mucous membrane. More particularly, the invention relates to the measuring and classifying of such changes by endoscopic observation.

A preferred field of application of the invention is the classification of skin anomalies such as, for examples, melanomata and melanotic changes in respect of benign, malignant or suspicious characteristics or properties. Other fields of application are the classification of anomalies of skins and mucous membranes of endoscopically observable cavitary organs, such as the cesophagus, the stomach, the duodenum, the colon, the rectum, the bronchial system, the cavities of the throat, nose and ears as well as the bladder. Such anomalies may be, for instance, diverticula, adenoids, carbuncles, tumor infiltrations and tumor compressions.

Malignant melanomata and the like may at the present time be identified with certainty in their late stage, when they are extremely dangerous and usually fatal. In their early stage, they may, however, be successfully treated in a relatively simple manner. At this point in time it is, however, very difficult to identify or diagnose them. That is to say, it is difficult at this time to classify skin anomalies as malignant or benign melanoma. It is the task, therefore, of the treating physician to classify skin anomalies into a group of harmless skin anomalies (benign melanomata and other benign non-melanocytic skin changes) and a group of unambiguously malignant or subjectively suspicious melanomata. It is possible, of curse, to observe or diagnose several skin anomalies for any one person. To ensure that not a single melanoma is overlooked the treating physician must, at the slightest suspicion of malignancy, classify the patient as one to be treated further.

This kind of approach will of necessity lead to a very large group of patients who are at risk. This, in turn, results in the motivation to improve the diagnosis by the use of commonly available means to allow qualitatively excellent classifications of skin anomalies, especially of melanomata, even by a physician who does not have the requisite expertise to classify merely on the basis of visual data.

2. The State of the Prior Art

For the registration of skin anomalies and melanomata in particular it has been known to irradiate a two-dimensional test area of the skin by light in a first range of wavelengths suited to generate fluorescence and in the wavelength range of the fluorescent light to generate an image of the test area (see German patent DE 4,026,821 A1). However, an evaluation of the fluorescent image by itself is not sufficiently reliable for classification in view of the fact that the topology of the skin surface in the test area exerts a strong influence on the intensity distribution of the fluorescent image. As disclosed by DE 4,026,821 A1, the influence of the topology of the skin surface is to be reduced. To this end the test area is irradiated by light of a second range of wavelengths, and a reference image is formed with light of the second wavelength range. By then scaling the values of intensity of the fluorescent image to those of the reference image, an image may be obtained in which the changes in intensity caused by the topology are substantially eliminated and which is suitable for diagnosis by the treating physician.

Also, a fluorescent diagnosis device suitable for testing tumors in the internal wall of body cavities is known from German patent DE 198 19 516 A1. Basically, the device consists of an endoscope and a camera for providing images of the test area on a monitor as well as of a spectrometer for spectrometrically examining the fluorescent light generated by selected sites of the test area. This diagnostic device does not, however, process the image to simplify the task of the diagnosing physician. Furthermore, the topology of the tested tissue area has no effect upon the diagnosis.

WO9747235 discloses a diagnostic system and method suitable for quantifying the visual appearance of skin anomalies, such as melanomata, by digitally analyzing images of the skin. In a preferred embodiment, the system includes a high-resolution digital camera, a computer, a data storage as well as a color monitor. The camera generates an image of the skin area to be examined, as well as, close to the area of the skin to be examined, of a longitudinal scale and of a color scale. This makes it possible to scale color values and spacings in the image independently of illumination and exposure conditions. The computer defines and subsequently displays on the monitor, the external contour of dark skin areas. Also, characteristic values of the color of the examined skin area as well as two-dimensional geometric characteristic values such as size and asymmetry of the external contour of dark skin areas may be derived, and characteristic values and images generated at different times may be compared, by the diagnostic system. Even though the diagnostic system of WO9747235 provides different characteristic values of geometry and color, an identification of skin anomalies is nevertheless strongly dependent upon the experience of the examining physician.

For rendering the classification of skin anomalies more objective and for automating it, it is known for purposes of classification to process difference characteristics of skin anomalies such as shape and color in color images of a skin area by a neuronal net (Ercal, F.: et al., IEEE Transactions on Biomedical Engineering, (1994 September) 41(9) 837-45). For classification, the method strongly relies upon the processing of characteristic values of color as well as upon the asymmetry and irregularities of the marginal contour of a skin anomaly. The method does not make use of the size or the diameter of a skin anomaly, or of their changes over time, as characteristic values during the classification process, and it requires a very precise definition of the margins of skin anomalies which for that reason is performed by a dermatologist.

Another example of the use of a neuronal net for classifying tumors in ultrasound images has been described in U.S. Pat. No. 5,260,871. In this case, characteristic values are extracted from the ultrasound images and input into the neuronal net which performs a classification. This solution, too, does not evaluate data relating to the three-dimensional structure of a suspicious area of tissue.

An essential disadvantage of known solutions resides in the fact that no data about the three-dimensional structure of suspicious tissue areas is being evaluated and used for purposes of classification. It has, however, been shown that for evaluating melanoma examinations, three-dimensional structure and growth in height are important criteria for determining the property of melanomata.

### OBJECTS OF THE INVENTION

Accordingly, it is an important object of the invention to provide a method and an apparatus suitable for measuring optically observable changes of skin and mucous membrane and which include the three-dimensional structure of such changes and their changes over time as important classification characteristics.

It is another object of the invention to measure and classify skin anomalies such as melanomata and melanoma-like symptoms by taking into consideration their three-dimensional and temporal changes.

### BRIEF SUMMARY OF THE INVENTION

As used herein, the term “skin” is intended to connote not only the external skin but also mucous membranes of a person or animal.

In accordance with a currently preferred embodiment the invention provides for a method of measuring and classifying optically observable changes in skin by, firstly, taking and recording at least one image of a change in the skin suitable for defining the three-dimensional structure and color structure and at least one reference image of at least the vicinity of the change, by, secondly, computing the surface measurements of the changes in skin consisting of the three-dimensional coordinates of the changes and of their associated color values scaled to the reference image and of, optionally, computing the changes over time, by, thirdly, performing a classification, preferably by means of a neuronal net, on the basis of the computed surface measurements together, optionally, with other data relevant to a patient such as age, type of skin, allergies, dermatoscopic or sonographic diagnoses, by, thirdly, performing another classification, particularly in a case which defies certain classification, on the basis of the previously obtained data but differing from the previous classification by the use of a modified algorithm and/or by using a selection of comparative data, preferably evaluated by experts, stored in at least one data base, and by, fourthly, issuing the results of the classification yielded by the third and fourth operations.

The apparatus in accordance with the invention preferably includes a measuring head or cameras for generating at least two photogrammetrically evaluatable images of changes in skin and a pattern projector for projecting onto the area of the skin to be examined a pattern suitable for detecting three-dimensional data of the area, at least one of the cameras being suitable for recording as reference images for defining a reference color images of an area of healthy skin in the vicinity of the changed skin, a first computer for preparing, processing and storing the images generated by the measuring head and, more particularly, for computing surface data of a change consisting of the three-dimensional coordinates of the change and the associated color values scales to the reference image, and for computing changes over time of these data, a second computer for executing a first classification of any changes on the basis of the surface measurement data and, optionally, of the stored relevant data relating to a patient such as age, type of skin, allergies, dermatoscopic or sonographic diagnoses, etc., optionally, a third computer for performing, with a modified algorithm and/or a selection from a data base of comparative data evaluated by experts, a further classification, particularly in the case of defying certain classification, on the basis of the surface measurement data and, optionally, of the stored relevant data relating to the patient.

The method of the invention is based upon the concept of generating in intervals of times appropriate for a diagnosis and by means of suitable recording techniques, such digital images of changes of the skins to be examined and optically observable which can be evaluated in respect of the three-dimensional structure of the depicted subject as well as of its color structure. A recording technique suitable for this purpose is known from the present inventors'German patent 196 23 172.

In a first operational step, the surface measurement data of changes in the skin and mucous membrane are computed from the digital images. The data consists of the three-dimensional coordinates and the associated color values scaled to the reference image. Optionally, the changes over time of the changes are additionally computed.

In a second operational step, the surface measurement data are evaluated, preferably by a neuronal net, by a process of classification. The result will be a classification into, for instance, benign, suspicious or malignant changes.

If it is not possible to obtain a certain classification on the basis of the image material provided in accordance with the invention and the surface measurement data derived therefrom, a third operational step will be performed in accordance with another basic concept of the invention. The third operational step comprises a classifying process differing from the previous one by a modified algorithm and/or by the use of a selection of comparative data, preferably evaluated by experts, stored in at least one data base.

In this data base, evaluated histories of diseases are documented by images recorded at different dates and by three-dimensional data as well as by data relevant to the disease. It is of utmost importance that the records of these histories of diseases have been classified with a very high degree of certainty, frequently by histological examinations. Instead of the classification of the changes in skin and mucous membrane, or in addition to the classification, an artificial neuronal net connected to the data base may can select comparative material from the data base, i.e., histories of diseases of changes in skins and mucous membranes the physical properties and appropriate relevant data of which are comparable to the changes in the skin and or mucous membrane to be examined. From the selected comparative data suitable images including evaluations which are similar to the input data may be selected and returned to the treating physician.

Execution of the operational steps is possible in the following variations:

all operational steps are being performed on a local computer;

the first and the second operational step are performed on a local computer and the third operational step is performed by a central computer;

the first operational step is performed on a local computer and the second and third operational steps are performed on a central computer;

all of the operational steps are performed on a central computer.

In accordance with a further advantageous embodiment of the inventive method at least one of the data bases containing comparative data evaluated by experts is disposed and maintained on the central computer, the local computer having access to the central computer.

The apparatus in accordance with the invention comprises two major components: A measuring head for generating at least to photogrammetrically evaluatable images of the changes in the skin to be examined and classified and at least one computer connected to the measuring head by way of data lines or integrated into the measuring head.

The measuring head is provided with at least two calibrated cameras, hereinafter sometimes referred to as camera group, for generating digital images of the area to be examined, and with a pattern projector for projecting onto the area to be examined at least one pattern suitable for detecting three-dimensional data of the said area. The camera group is adjusted such a surface of appropriate size may be measured by it. At least one of the cameras of the camera group is adapted for taking color images, this camera or, optionally, a further camera, being additionally provided for taking, as a reference image for defining a reference color, an image of an inconspicuous area in the vicinity of the change in the skin. The measuring head is movable and may be positioned at an appropriate location.

The at least one computer connected to the measuring head by way of data lines is provided with means for preparing, processing and storing the digital images of the skin and mucous membrane changes generated by the measuring head. Such means may include means for computing data of the three- and two-dimensional structures of the changes and their color structures relative to the reference image as well as of the temporal changes of the said data. The computer is additionally provided with means for carrying out a classification on the basis of the previously mentioned detected data and, if necessary, the relevant data relating to a patient such as age, type of skin, allergies, dermatoscopic or sonographic diagnoses etc. A neuronal net may be used to provide such classification.

For use in connection with a case which cannot be classified with certainty, the computer is preferably provided with means for carrying out a Attorney Docket 010458 9 further classification with a modified algorithm and/or a selection of comparative data evaluated by experts, on the basis of the previously detected data and, if necessary, the relevant patient-related data stored in a data base.

In an advantageous embodiment of the device in accordance with the invention there are provided at least two computers interconnected by data lines. At least one of the computers is a local one and at least one other computer is a central one. In such a case only the central computer is provided with the means applied in a case which cannot be classified with certainty such as, in particular, the data base of comparative data evaluated by experts.

### DESCRIPTION OF THE DRAWING

The novel features which are considered to be characteristic of the invention are set forth with particularity in the appended claims. The invention itself, however, in respect of its structure, construction and lay-out as well as manufacturing techniques, together with other objects and advantages thereof, will be best understood from the following description of preferred embodiments when read in connection with the appended single drawing which is a flow diagram schematically depicting the cooperation between the operational steps in accordance with the invention.

The example shown relates to the classification of skin anomalies known as melanoma and melonocytic changes into suitable categories such as, for instance, benign, suspicious or malignant.

### DETAILED DESCRIPTION OF THE PREFERRED EMBODIMENTS

The measuring head comprises a camera group of two calibrated color cameras for generating digital images and a pattern projector. Where necessary, the pattern projector makes it possible to illuminate skin anomalies by unstructured light, i.e. at a substantially constant light intensity, with one light pattern or with a plurality of sequential light patterns. The lenses and the positions of the camera group and of the pattern projector are chosen so that a surface measuring about 4×4 cm may be measured by them. A surface, e.g., a ring, with reference colors constitues a further component of the measuring head. This is an accessory for efficiently comparing results of measurements provided by different measuring heads.

To obtain the images necessary for determining the measurment data of skin anomalies the following procedure is deemed appropriate:

Initially a color image is recorded of a healthy area of skin in the vicinity of a skin anomaly. This is a reference image and serves to define the actual basic skin color (basic complexion) of a patient. The color data of a skin anomaly may be scaled relative to the reference image.

Thereafter, the measuring head is positioned such that the skin anomaly is preferably positioned in the central measuring range of the camera group. In the case of two or more closely adjacent skin anomalies it may be useful to deviate from the rule such that these skin anomalies are possibly detected by a single position of the measuring head. A color image, hereinafter sometimes referred to as a light image, in which the skin anomaly is illuminated with unstructured light may then be taken by at least one camera of the camera group. This image serves to define the actual color values of the skin change.

Furthermore, without changing the position of the measuring head, images are recorded in which one or, sequentially, several appropriate light patterns are projected onto the skin anomaly by the pattern projector. Such light patterns are indispensable to the process.

The recorded digital images or image sequences form the basis of the computation of the data relating to the three- and two-dimensional structures of the skin anomaly and its color structure relative to the reference image. To this end, a local computer, proceeding from the recorded digital images, computes in a first process or operational step surface measurement data of the skin anomaly. The surface measurement data consist of the three-dimensional coordinates and associated scaled color values of the surface of the skin anomaly.

The images or image sequences of the skin anomaly taken with the light patterns are used to define the three-dimensional coordinates. Known or novel algorithms used in image processing are used for the computation. The algorithms are based on the definition of corresponding pairs of pixels by matching processes or by correlation algorithms.

The light image and the reference image are used to define the scaled color values of the skin anomaly. The color values of the light image are affected by the activity of the skin anomaly as well as by the basic complexion of the skin (depending on a seasonally conditioned degree of tanning). To minimize the effect of the last-mentioned affect scaling relative to the reference image is useful. Scaling may be carried out in the conventionally (use of an offset and lightness correction) or by the neuronal net manner. The result will be scaled color values. Correlating the scaled color values to the three-dimensional coordinates is simple since both data are defined on the basis of images from the same cameras in the same positions.

The surface measurement data of the skin anomaly determined in this manner are subjected in a second operational step also performed on a local computer to a first classification process by means of a neuronal net. This results in a correlation of appropriate categories, e.g., benign, malignant or suspicious. This result and the surface measurement data obtained are well suited for documentation and archiving in a patient data base. If measurements are taken at different times, the relevant changes of the surface measurement data of the skin anomaly can be determined from the documentation.

In order further to improve the quality of classification a telematic system may optionally be utilized. In such a system the surface measurement data of the skin anomaly measured by the treating physician are transferred to a central computer and correlated to or compared with relevant patient related data obtained elsewhere, such as age, type of skin, allergies, dermatoscopic or sonographic diagnoses and so forth.

A third operational or process step may then be carried out on the central computer. The third step consists of a further classification based on modified algorithms and/or a selection of suitable comparative data from at least one data base.

The data base may be a central data base stored in a central computer. Alternatively or additionally, the data base may be an external data base which may for instance be accessible through the Internet.

Such data banks contain evaluated histories of diseases documented by images and surface measurement data recorded at different times as well as relevant patient-related data (described supra) relevant to the disease. It is decisive that these disease histories have been classified with a high degree of Attorney Docket 010458 13 certainty, often by histological examinations.

On the basis of data stored in the mentioned data bases the artificial neuronal net or another suitable algorithm of the central computer will detect skin anomalies the surface measurement data of which are sufficiently similar to the skin anomaly measured by the treating physician and transferred to the central computer. The patient-related data are also considered. In this manner, it is possible to obtain suitable comparative data such as evaluated images and optional additional data such as histological diagnoses and other data of interest from the history of disease. They are returned to the treating physician. They serve to improve the result of the classification.

Another telematic application resides in consulting an expert to whom the surface measurement data, the result of the classification and relevant patient-related data are submitted by data lines. Such consultation may be carried out in the manner of video conferencing.

Further possibilities of use of the inventive method and apparatus arise in connection with the following problems:

The measuring head may be utilized for augmenting diagnoses of other diseases in the field of dermatology. A typical application resides in the measuring and evaluation of skin rashes. Another application is the measuring changes of color and height caused by allergy tests. Precise documentation is made possible by the measuring head.

For documenting and evaluating the healing progress of wounds measurements may be very useful. To this end, two-dimensional and three-dimensional data as well as the color structure are measured at one or more points in time. Analogously, measurements may be taken of skin burns where a precise definition of the degree of burn is of importance.

Radiation treatments may also result in changes in the skin color of a patient. Where such changes are measured it is possible to arrive at conclusions as to the radiation sensitivity of the patient.

The measuring head may also be used for measuring and evaluating skin creases or wrinkles (primarily in a face). This may be important to the cosmetics industry in connection with determining, for instance, the effectiveness of skin creams and lotions.

What is claimed is:

1. A method of measuring and classifying in predetermined categories optically observable changes in skin and mucous membrane, comprising the steps of:
1. taking at at least one instant in time at least one digital image of a change in a skin suitable for determining the three-dimensional structure and color structure of the change and at least one digital reference image of at least the vicinity of the change;
2. computing surface measurement data comprising three-dimensional coordinates of the change and color values scaled to the reference image on the basis of the digital image and digital reference image;
3. carrying out a first process of classification on the basis of the surface measuring data;
4. carrying out a further process of classification differing from the first process by at least one modified algorithm;
5. Issuing the results of the first and second classification processes.

2. The method of claim 1, further comprising the step of storing the digital image and the digital reference image in a memory.

3. The method of claim 1, wherein the surface measurement data are additionally computed on the basis of temporal changes of the skin.

4. The method of claim 1, wherein the first process of classification is additionally optionally carried out on the basis of externally collected relevant patient-related data.

5. The method of claim 4, wherein the patient-related data includes at least one of age, type of skin, allergies, dermatoscopic diagnoses and sonoghraphic diagnoses.

6. The method of claim 1, wherein the further process is carried out by an artificial neuronal net.

7. The method of claim 1, wherein the further classification process includes the use of selected comparative data.

8. The method of claim 7, wherein the selected comparative data includes data evaluated by experts.

9. The method of claim 7, wherein the selected comparative data is stored in a memory.

10. The method of claim 1, further including the step of projecting at least one light pattern on the change during taking of the digital image.

11. The method of claim 1, wherein the digital image is taken with unstructured light.

12. The method of claim 1, wherein at least one step is performed on a local computer.

13. The method of claim 1, wherein at least one step is performed on a central computer.

14. The method of claim 7, wherein the comparative data is stored on at least one data bank in a central computer.

15. The method of claim 7, wherein the comparative data is stored in at least one data bank in a local computer.

16. The method of claim 2, wherein the memory is in a local computer connected to a central computer in which comparative data relevant to the skin change is stored.

17. An apparatus for measuring and classifying an optically observable change in a skin, comprising:
1. a measuring head comprising at least two calibrated cameras for generating at least two photogrammetrically evaluatable digital images of the change and a pattern projector for projecting a suitable pattern onto an area of the skin to be measured, at least one of the cameras being adapted for taking a color reference image of healthy skin in the vicinity of the change;
2. a first computer for preparing, processing and storing the digital images and for computing surface measurement comprising three-dimensional coordinates and associated color values scaled to the reference image; and
3. a second computer for carrying out a classification process on the basis of at least the surface measuring data computed by the first computer.

18. The apparatus of claim 17, further comprising a third computer for carrying out a further classification process on the basis of the surface measuring data by one of a modified algorithm and select data evaluated by an expert from a data bank.

19. The apparatus of claim 17, wherein the first and second computers are an integrated unit.

20. The apparatus of claim 18, wherein at least one of the first, second and third computers is a local computer and at least one other of the first, second and third computers is a central computer.
